(19) United States
(12) Patent Application Publication
(10) Pub. No.: US 2017/0281892 A1
(43) Pub. Date: Oct. 5, 2017

(54) TRACHEA CANNULA

(71) Applicant: Zhihui HUANG, Guangdong (CN)

(72) Inventor: Zhihui HUANG, Guangdong (CN)

(21) Appl. No.: 15/512,799

(22) PCT Filed: Mar. 25, 2015
(86) PCT No.: PCT/CN2015/075009

§ 371 Date: Mar. 20, 2017

(30) Foreign Application Priority Data
Sep. 18, 2014 (CN) 201420538397.5

(51) Int. Cl. A61M 16/04

(52) U.S. Cl. CPC A61M 16/0488; A61M 16/0434; A61M 2205/583; A61M 2205/587; A61M 2205/8206

(57) ABSTRACT

A trachea cannula comprises a cannula body (1) and an inflatable bag (2), the cannula body is hollow structured and comprises a front end inserted into oral cavity of human and a rear end outside the oral cavity, and the front end of the cannula body is configured with an opening (6) which is disposed at a head of the front end. The trachea cannula further comprises a visible light source (3), a switch (5) for controlling the visible light source, and a battery (6) supplying power to the visible light source, and both the inflatable bag and the visible light source are secured at the front end of the cannula body; the switch, the battery and the visible light source are connected together in series via wires, forming a closed circuit; the distance from the visible light source to the opening is equal to the distance from a middle point between the third tracheal ring and the fourth tracheal ring to a position 1.5 to 2 cm from tracheal bifurcation at the trachea. Such intubation for the tracheal facilitates the medical staff to determine whether the trachea cannula is located at a correct position conveniently and quickly based on whether the visible light source is placed a position between the third tracheal ring and the fourth tracheal ring, without any harm to human body.

### FIELD OF TECHNOLOGY

The present invention relates to a technical filed of medical devices, particularly to a trachea cannula.

### BACKGROUND

It is usually needed to insert a trachea cannula into patient's body according to requirements of surgery or nursing, and the position of an inserted trachea cannula is significant. The opening of the trachea cannula must be located at a position 1.5 to 2 cm from the bifurcation of trachea. The trachea cannula would not work well if its opening is located too far from the bifurcation of trachea, whereas the trachea cannula will be inserted into a bronchia in case of its deep insertion. In order to ensure a proper position of the inserted trachea cannula, three measures, i.e., determination by 1) experience, 2) x-ray and 3) auscultation of lungs, are usually applied. However, these three inconvenient measures may delay rescue to the patient due to long time intubation, especially it will harm human body if x-ray is applied. Therefore, it is a technical problem urgently to be resolved in the medical application to insert the trachea cannula into trachea of human quickly and accurately, which is vital for the treatment of patients. Urgent trachea intubation technique has become an important measure in the cardio-pulmonary resuscitation and the rescue of critical patients concomitant with respiratory dysfunction.

### SUMMARY

An aspect relates to a trachea cannula configured with a visible light source, wherein the visible light source can emit light penetrating the skin when it is located between the third trachea ring and the fourth trachea ring, whereby the medical staff can determine whether the trachea cannula is inserted into a correct position conveniently and quickly, based on the judgment as to whether the visible light source is being placed at a position between the third trachea ring and the fourth trachea ring, without any harm to the human body.

The present invention provides following technical solution.

A trachea cannula comprises a cannula body and an inflatable bag, the cannula body is hollow structured and comprises a front end inserted into oral cavity of human and a rear end outside the oral cavity, and the front end of the cannula body is configured with an opening which is disposed at a head of the front end of the cannula body. The trachea cannula further comprises a visible light source, a switch for controlling the visible light source, and a battery supplying power to the visible light source, and both the inflatable bag and the visible light source are secured at the front end of the cannula body. The switch, the battery and the visible light source are connected together in series via wires, forming a closed circuit, and the distance from the visible light source to the opening is equal to the distance from a middle point between the third tracheal ring and the fourth tracheal ring to a position 1.5 to 2 cm from a tracheal bifurcation at the trachea.

The cannula body is bendable, and the visible light source is secured at the inward side of the front end of the cannula body.

There are a plurality of visible light sources secured annularly at an outer wall of the front end of the cannula body.

The visible light source is a LED light.

The visible light source emits red light.

The wires are embedded in the inside of cannula wall of the cannula body.

The opening is in apex angle.

A scale is marked on a surface of the cannula body.

The present invention has the following advantages.

The trachea cannula of the present invention is configured with a visible light source, the distance from the visible light source to the opening of the trachea cannula is equal to the distance from the middle point between the third tracheal ring and the fourth tracheal ring to a position 1.5 to 2 cm from the tracheal bifurcation at the trachea. The position between the third tracheal ring and the fourth tracheal ring is closest to the human skin, thereby the visible light source emits light penetrating the skin therefrom, further the third tracheal ring and the fourth tracheal ring can be perceived due to their protrusion shapes at the neck. When the trachea cannula is inserted into the human trachea and the visible light source is located between the third tracheal ring and the fourth tracheal ring, the opening of the trachea cannula is just located at an entrance of the bronchus, whereby medical staff can determine whether the trachea cannula is located at a correct position conveniently and quickly based on the judgment as to whether the visible light source is being placed at the position between the third tracheal ring and the fourth tracheal ring, according to a light emitted from the visible light source together with the perceived position of the third tracheal ring and the fourth tracheal ring, without any harm to human body.

### BRIEF DESCRIPTION

The present invention will be described hereinafter in details with reference to embodiment and figures.

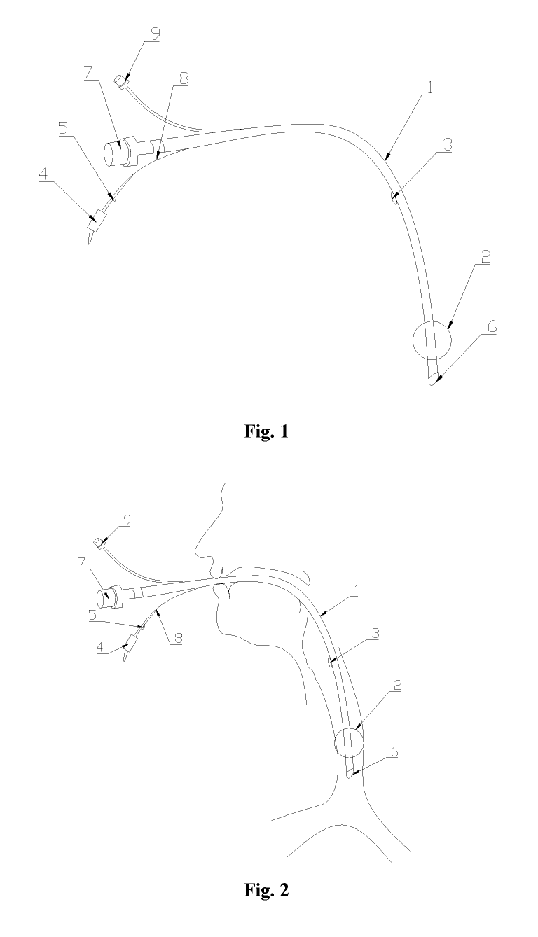

FIG. 1 is a structural schematic diagram of the trachea cannula of the present invention;

FIG. 2 is a schematic diagram showing the position of the trachea cannula of the present invention, when it is inserted into trachea of human.

### DETAILED DESCRIPTION

The present invention will be described hereinafter with reference to the following embodiment and figures.

As shown in FIG. 1, the present invention provides a trachea cannula, comprising a cannula body 1, an inflatable bag 2, a visible light source 3, a switch 5 for controlling the visible light source 3, and a battery 4 supplying power to the visible light source 3. The cannula body 1 is hollow structured, and comprises a front end inserted into oral cavity of human and a rear end outside the oral cavity. The front end of the cannula body is configured with an opening 6, the inflatable bag 2 and the visible light source 3, wherein the opening 6 is configured at the head of the front end of the cannula body. The switch 5 and the battery 4 are connected with the visible light source 3 in series via wires 8, forming a closed circuit. The distance from the visible light source 3 to the opening 6 is equal to the distance from the middle point between the third trachea ring and the fourth trachea ring to a position 1.5 to 2 cm from a tracheal bifurcation at the trachea. When the trachea cannula is inserted into the human body, and the opening 6 is located at a position 1.5-2 cm from the tracheal bifurcation in the trachea, the visible light source is located between the third trachea ring and the fourth trachea ring.

The trachea cannula can be classified into several models according to human structure in their different ages, and the distance between the visible light source 3 and the opening 6 corresponds to the model of the trachea cannula. The distance between the visible light source 3 and the opening 6 is equal to the distance from the middle point between the third trachea ring and the fourth trachea ring to a position 1.5 to 2 cm from the tracheal bifurcation at the trachea. When the cannula body 1 is inserted into the trachea of human body, and the visible light source 3 is placed at a position between the third trachea ring and the fourth trachea ring, the light source 3 emits light penetrating the skin and people can see the light from the visible light source 3 through the skin, as the visible light source 3, about 1.5 cm from the skin, is nearest to human skin at the whole trachea. Besides, the third trachea ring and the fourth trachea ring can be perceived due to their protruding shape at the neck. When the visible light source 3 is inserted into the position between the third trachea ring and the fourth trachea ring, the opening 6 of the trachea cannula will be just located at a position 1.5 to 2 cm from the tracheal bifurcation, thereby medical staff can determine whether the trachea cannula is located at a correct position based on the judgment as to whether the visible light source is being placed at the position between the third tracheal ring and the fourth tracheal ring, according to a light emitted from the visible light source 3 together with the perceived position of the third tracheal ring and the fourth tracheal ring.

The cannula body 1 is bendable, and the visible light source 3 is secured at the inward side of the front end of the cannula body. When the cannula body is inserted into the trachea, the visible light source 3 is placed at a nearest position to the skin, thus the visible light source 3 can be seen clearly. Further, the visible light sources may be configured annularly around the external wall of the cannula body 1, which can enhance the luminous intensity and prevent from a situation that the medical staff will be unable to observe the light in case the deviation of the visible light source 3 since the cannula body 1 turns after it is inserted.

The visible light source 3 may be LED light which has a small size and various shapes, thus the visible light source 3 can be small and be shaped properly. Preferably, the visible light source 3 emits red light, which is sensitive for human eyes, and can be observed easily.

The opening 6 of the front end of the cannula body 1 is in apex angle, which facilitates the medical staff to insert the cannula body 1 into human trachea.

A respirator connector 7 and an inflatable bag connector 9 are configured at the rear end of the cannula body, wherein the respirator connector 7 is used to connect the respirator to supply oxygen to human body, and the inflatable bag connector is used to connect an inflatable apparatus in order to inflate the inflatable bag.

The wires 8 are embedded in the inside of cannula wall of the cannula body 1, facilitating the medical staff to insert the cannula body 1 into human trachea.

A scale is marked on the surface of the cannula body 1, whereby the medical staff can observe the scale to determine the position of the inserted cannula body 1 approximately, and make a further accurate positioning for the inserted cannula body 1 by observing the light emitted from the visible light source 3.

The trachea cannula can be inserted into human trachea by following steps:
(1) Inserting the trachea cannula into human oral cavity slowly, while opening the switch 5, and observing the position between the third tracheal ring and the fourth tracheal ring;
(2) Stopping the insertion of the trachea. cannula if a light is visible from the position between the third tracheal ring and the fourth tracheal ring;
(3) Inflating the inflate bag 2 till the trachea is sealed up;
(4) Securing the rear end of the cannula body to the human face.

The trachea cannula and its positioning method of the present invention are provided according to the principles of anthropotomy and optics. Considering the position between the third tracheal ring and the fourth tracheal ring is closest to the human skin and thus a visible light can be penetrated therefrom, further the third tracheal ring and the fourth tracheal ring can be perceived without any instrument. Therefore the visible light source is secured at the trachea cannula, and the distance from the visible light source to the opening of the trachea cannula is configured equal to the distance from the middle point between the third trachea ring and the fourth trachea ring to a position 1.5 to 2 cm from the tracheal bifurcation at the trachea. When a light is observed between the third tracheal ring and the fourth tracheal ring after the trachea cannula is inserted into the human trachea, it can be determined that the visible light source is positioned at the position between the third tracheal ring and the fourth tracheal ring, and the opening of the trachea cannula is located at a place 1.5 to 2 cm from the tracheal bifurcation at the trachea. Such method is simple and convenient, making an accurate trachea intubation, ensuring a one-off success in intubation without other auxiliary instrument and any harm to human body.

It should be understood that the embodiment described hereinbefore is merely preferred embodiment of the present invention and not for purposes of any restrictions or limitations on the invention. Although the present invention are described here in details with reference to the embodiment, any non-substantive, obvious, equivalent alterations or improvement by the technician of this technical field according to the present invention may be incorporated into ambit of claims of the present invention.

1. A trachea cannula, comprising:
a cannula body; and
an inflatable bag,
wherein the cannula body is hollow and comprises a front end inserted into an oral cavity of a subject and a rear end that remains outside the oral cavity,
wherein the front end of the cannula body is configured with an opening disposed at a head thereof,
wherein the trachea cannula further comprises:
a visible light source;
a switch configured to control the visible light source; and
a battery configured to supply power to the visible light source;
wherein both the inflatable bag and the visible light source are secured at the front end of the cannula body;
wherein a closed circuit is formed by connecting the switch, the battery and the visible light source together in series via wires, and
wherein a distance from the visible light source to the opening is equal to a distance from a middle point between a third tracheal ring and a fourth tracheal ring to a position 1.5 to 2 cm from a tracheal bifurcation of the subject's trachea.

2. The trachea cannula according to claim 1, wherein the cannula body is bendable, and the visible light source is secured at an inward side of the front end of the cannula body.

3. The trachea cannula according to claim 1, further comprising a plurality of visible light sources secured annularly at an outer wall of the front end of the cannula body.

4. The trachea cannula according to claim 1, wherein the visible light source is an LED light.

5. The trachea cannula according to claim 4, wherein the visible light source emits red light.

6. The trachea cannula according to claim 1, further comprising wires embedded in an inside of a wall of the cannula body.

7. The trachea cannula according to claim 1, wherein the opening forms an apex angle.

8. The trachea cannula according to claim 1, wherein a scale is marked on a surface of the cannula body.

9. The trachea cannula according to claim 2, wherein the visible light source is an LED light.

10. The trachea cannula according to claim 9, wherein the visible light source emits red light.

11. The trachea cannula according to claim 3, wherein the plurality of visible light sources are each an LED light.

12. The trachea cannula according to claim 11, wherein the plurality of visible light sources emit red light.
